(19) United States
(12) Patent Application Publication
(10) Pub. No.: US 2017/0128341 A1
(43) Pub. Date: May 11, 2017

(54) DECOLORIZATION OF DYED KERATINIC FIBERS

(71) Applicant: Henkel AG & Co. KGaA, Duesseldorf (DE)

(72) Inventors: Juergen Schoepgens, Schwalmtal (DE); Burkhard Mueller, Duesseldorf (DE)

(73) Assignee: Henkel AG & Co. KGaA, Duesseldorf (DE)

(21) Appl. No.: 15/348,510

(22) Filed: Nov. 10, 2016

(30) Foreign Application Priority Data
Nov. 11, 2015 (DE) 10 2015 222 215.2

(51) Int. Cl. A61K 8/46; A61K 8/41; A61K 8/34; A61K 8/23; A61Q 5/08; A61K 8/55; A61Q 5/12; A61K 8/22

(52) U.S. Cl. CPC A61K 8/46; A61Q 5/12; A61K 8/41; A61K 8/22; A61K 8/23; A61Q 5/08; A61K 8/463; A61K 8/55; A61K 8/342; A61K 2800/88; A61K 2800/882

(57) ABSTRACT

A multi-component packaging unit (kit-of-parts) for the reductive decolorization of dyed keratinic fibers, which unit includes, packaged separately from one another,
a container (A) containing a solid or powdered cosmetic agent (a) and
a container (B) containing an aqueous cosmetic agent (b),
   wherein
   the agent (a) in container (A)
   (a1) includes one or more sulfinic acid derivatives from a specific group and the agents (a) and (b) are substantially free from metal salts.

Also, a method for the reductive decolorization of dyed keratinic fibers with use of the multi-component packaging unit.

### FIELD OF THE INVENTION

The present invention generally relates to cosmetics, and more particularly relates to multi-component packaging units (kits-of-parts) for the reductive decolorization of dyed keratin fibers, said units comprising the containers (A) and (B) packaged separately from one another.

### BACKGROUND OF THE INVENTION

Preparations for tinting and dyeing hair are an important type of cosmetic agent. They can be used to lightly or heavily nuance the natural hair color in accordance with the wishes of the person in question, to attain a completely different hair color or to cover undesirable shades of color, for example grey shades. Conventional hair colorants, depending on the desired color or permanence of the coloration, are formulated either on the basis of oxidation dyes or on the basis of substantive dyes. Combinations of oxidation dyes and substantive dyes are also often used to attain specific nuances.

Colorants based on oxidation dyes lead to bright and permanent color shades. However, they require the use of strong oxidizing agents, such as hydrogen peroxide solutions. Such colorants contain oxidation dye precursors, or what are known as developer components and coupler components. The developer components form the actual dyes under the influence of oxidizing agents or atmospheric oxygen, either among themselves or with coupling to one or more coupler components.

Colorants based on substantive dyes are often used for temporary coloring. Substantive dyes are dye molecules which are drawn directly onto the hair and do not require any oxidative process to form the color. Important representatives of this class of dyes include, for example, triphenylmethane dyes, azo dyes, anthracine dyes, or nitrobenzene dyes, which can each carry cationic or anionic groups.

In all of these dyeing processes, it may be that the coloring must be reversed again wholly or partially for various reasons. A partial removal of the coloring is suggested for example when the color result on the fibers is darker than desired. On the other hand, a complete removal of the coloring may also be desired in some cases. By way of example, it is thus conceivable for the hair to be dyed or tinted in a specific nuance for a certain reason and then for the original color to be restored again after a few days.

Agents and methods for dye removal are already known in the literature. A method that has long been known from the prior art for reversing coloring is the oxidative decolorization of the dyed hair, for example with the aid of a conventional bleaching agent. In this process, however, the fibers can be damaged by the use of strong oxidizing agents.

Furthermore, reductive processes for dye removal have also already been described. By way of example, European patent application EP 1300136 A2 discloses methods for hair treatment in which the hair is dyed in a first step and is reductively decolorized again in a second step. Here, the reductive decolorization occurs as a result of the application of a formulation containing a dithionite salt and a surfactant. In WO 2008/055756 A2 the reductive decolorization of keratin fibers is performed by means of a mixture of a reducing agent and an absorption agent.

With use of reductive decolorizing agents, the decolorization takes place by reduction of the dyes located on the keratin fibers or hairs. As a result of the reduction, the dyes are generally converted into their reduced leukoforms. During this process, the double bonds present in the dyes are reduced, the chromophoric system of the dyes is thus interrupted, and the dye is converted into a colorless form.

A general problem with the reductive decolorizing agents known from the prior art lies in the fact that the dyed keratin fibers can indeed be decolorized initially by use of the reducing agent, however the color removal is not permanent. In particular in the case of oxidatively dyed hair, in which the coloring is produced by oxidation dye precursors of the developer and of the coupler type, colorings are obtained that have very good fastness properties in part. With use of the reductive decolorizing agent, these dyes are now reductively converted into uncolored compounds—these still remain on the hair, however, on account of similarly good fastness properties.

Once the reducing agent has been rinsed off and under the influence of atmospheric oxygen, these reduced forms can now be re-oxidized again little by little. A lesser or greater re-coloration takes place on account of this re-oxidation. This re-coloration generally does not correspond to the shade to which the keratin fibers were dyed previously, and instead may be unattractive at random points and is therefore even less desired by the user of the decolorizing agent. For this reason, agents for reductive color removal in which the above-described re-coloration does not occur are still sought.

Sulphurous compounds, such as cysteine or dithionite salts, are generally used as reducing agents, but have a strong sulphurous odor during application and are thus considered to be a significant odor nuisance for the user. Agents which have a more subtle smell are thus also sought.

It is therefore desirable to provide a decolorizing agent for the decolorization of dyed keratinic fibers which decolorizes dyed keratin fibers as completely as possible. The decolorization should be long lasting, and the decolorized keratin fibers should not suffer from any re-coloration, nuance shift, or post-darkening under the influence of atmospheric oxygen. The decolorizing agent should, in particular, demonstrate a good decolorization effect on the keratin fibers dyed previously with oxidative colorants on the basis of oxidation dye precursors of the developer and of the coupler type. In particular, the odor nuisance produced by the agent should be minimized or even avoided.

Furthermore, other desirable features and characteristics of the present invention will become apparent from the subsequent detailed description of the invention and the appended claims, taken in conjunction with this background of the invention.

### BRIEF SUMMARY OF THE INVENTION

A multi-component packaging unit (kit-of-parts) for the reductive decolorization of dyed keratinic fibers, which unit comprises, packaged separately from one another a container (A) containing a solid or powdered cosmetic agent (a) and a container (B) containing an aqueous cosmetic agent (b), wherein the agent (a) in container (A) contains one or more sulfinic acid derivatives from the group consisting of (H2N)(NH)C(SO₂H) formamidine sulfinic acid; HN(CH₂SO₂Na)₂disodium [(sulfinatomethyl)amino]methane sulfonate; HN(CH₂SO₂K)₂, dipotassium [(sulfinatomethyl)amino]methane sulfonate; HN(CH₂SO₂H)₂, [(sulfinomethyl)amino]methanesulfinic acid; N(CH₂SO₂Na)₃, trisodium [bis(sulfinatomethyl)amino]methane sulfonate; N(CH₂SO₂K)₃, tripotassium [bis(sulfinatomethyl)amino]methane sulfonate; N(CH₂SO₂H)₃, [bis(sulfinomethyl)amino]methanesulfinic acid; H₂NCH(CH₃)SO₂Na, sodium 1-aminoethan 1-sulfinate; H₂NCH(CH₃)SO₂K, potassium 1-aminoethan 1-sulfinate; H₂NCH(CH₃)SO₂H, 1-aminoethan-1-sulfinic acid; HN(CH(CH₃)SO₂Na)₂, disodium 1-[(1-sulfinatoethyl)amino]ethan-1-sulfinate; HN(CH(CH₃)SO₂K)₂, dipotassium 1-[(1-sulfinatoethyl)amino]ethan-1-sulfinate; HN(CH(CH₃)SO₂H)₂, 1-[(1-sulfinoethyl)amino]ethan-1-sulfinic acid; N(CH(CH₃)SO₂Na)₃, trisodium 1-[bis(1-sulfinatoethyl)amino]ethan-1-sulfinate; N(CH(CH₃)SO₂K)₃, tripotassium 1-[bis(1-sulfinatoethyl)amino]ethan-1-sulinate; and/or N(CH(CH₃)SO₂H)₃, 1-[bis(1-sulfinoethyl)amino]ethan-1-sulfinic acid, and the total content of all metal salts contained in the agent (a) from the group of magnesium salts, zinc salts, iron salts, copper salts, and calcium salts—in relation to the total weight of the agent (a)—lies at a value below 0.1% by weight, and the total content of all metal salts contained in the agent (b) from the group of magnesium salts, zinc salts, iron salts, copper salts, and calcium salts—in relation to the total weight of the agent (b)—lies at a value below 0.1% by weight.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has surprisingly now been found that the odor developing and occurring during the use of the reductive decolorizing agent can be practically completely suppressed when a multi-component packaging unit comprising two different agents (a) and (b) is used for the purpose of color removal. The agent (a) is packaged in solid or powder form and contains at least one reducing agent from a group of certain sulfinic acids (a1), and the agent (b) constitutes an aqueous carrier formulation. It could be found that both the solid/powdered agent (a) and the application mixture produced by mixing the agents (a) and (b) developed practically no odors.

These two agents (a) and (b) are to be used in methods for the reductive removal of color from dyed keratinic fibers, in particular human hair. A corresponding method constitutes a further subject of the present application. For the purpose of the reductive removal of color, the solid or powdered agent (a), which contains the sulfinic acid derivative, is mixed with the aqueous cosmetic carrier agent (b). During the mixing, the solid or powdered agent (a) dissolves. The application mixture produced in this way is applied to dyed keratinic fibers, is left on the keratin fibers for an application time of from 2 to 60 minutes, and is then rinsed out again.

A first subject matter of the present invention is a multi-component packaging unit (kit-of-parts) for the reductive decolorization of dyed keratinic fibers, which unit comprises, packaged separately from one another,
a container (A) containing a solid or powdered cosmetic agent (a) and
a container (B) containing an aqueous cosmetic agent (b),
wherein
the agent (a) in container (A)
(a1) contains one or more sulfinic acid derivatives from the group consisting of
   (H2N)(NH)C(SO₂H) formamidine sulfinic acid,
   HN(CH₂SO₂Na)₂, disodium [(sulfinatomethyl)amino]methane sulfinate
   HN(CH₂SO₂K)₂, dipotassium [(sulfinatomethyl)amino]methane sulfinate
   HN(CH₂SO₂H)₂, [(sulfinomethyl)amino]methanesulfinic acid
   N(CH₂SO₂Na)₃, trisodium [bis(sulfinatomethyl)amino]methane sulfinate
   N(CH₂SO₂K)₃, tripotassium [bis(sulfinatomethyl)amino]methane sulfinate
   N(CH₂SO₂H)₃, [bis(sulfinomethyl)amino]methanesulfinic acid
   H₂NCH(CH₃)SO₂Na, sodium 1-aminoethan-1-sulfinate
   H₂NCH(CH₃)SO₂K,potassium 1-aminoethan-1-sulfinate
   H₂NCH(CH₃)SO₂H, 1-aminoethan-1-sulfinic acid,
   HN(CH(CH₃)SO₂Na)₂, disodium 1-[(1-sulfinatoethyl)amino]ethan-1-sulfinate
   HN(CH(CH₃)SO₂K)₂, dipotassium 1-[(1-sulfinatoethyl)amino]ethan-1-sulfinate
   HN(CH(CH₃)SO₂H)₂, 1-[(1-sulfinoethyl)amino]ethan-1-sulfinic acid
   N(CH(CH₃)SO₂Na)₃, trisodium 1-[bis(1-sulfinatoethyl)amino]ethan-1-sulfinate
   N(CH(CH₃)SO₂K)₃, tripotassium 1-[bis(1-sulfinatoethyl)amino]ethan-1-sulinate and/or
   N(CH(CH₃)SO₂H)₃, 1-[bis(1-sulfinoethyl)amino]ethan-1-sulfinic acid and
the total content of all metal salts contained in the agent (a) from the group of magnesium salts, zinc salts, iron salts, copper salts, and calcium salts—in relation to the total weight of the agent (a)—lies at a value below 0.1% by weight, and
the total content of all metal salts contained in the agent (b) from the group of magnesium salts, zinc salts, iron salts, copper salts, and calcium salts—in relation to the total weight of the agent (b)—lies at a value below 0.1% by weight.

The multi-component packaging unit according to the invention comprises the containers (A) and (B) packaged separately from one another and containing, respectively, the agents (a) and (b). The agent (a) is present in solid form or in powder form and contains at least one reducing agent from the group of sulfinic acids (a1). The agent (b) is an aqueous carrier formulation. The ready-to-use decolorizing agent is produced by mixing the two agents (a) and (b)—i.e. by mixing the reducing agent-containing agent (a) with the carrier (b).

Keratinic fibers, keratin-containing fibers or keratin fibers are to be understood to mean furs, wools, feathers and in particular human hair. Although the agents according to the invention are primarily suitable for lightening and coloring keratin fibers or human hair, there is nothing in principle standing in the way of a use in other fields as well.

The term “dyed keratinic fibers” is understood to mean keratin fibers that have been dyed using conventional cosmetic colorants known to a person skilled in the art. In particular, “dyed keratinic fibers” are understood to mean fibers which have been dyed using the oxidative colorants known from the prior art and/or using substantive dyes. Express reference is made in this regard to the known monographs, for example Kh. Schrader, Grundlagen and Rezepturen der Kosmetika (Basic Principles and Formulations of Cosmetics), 2^{nd} edition, Hüthig Buch publishers, Heidelberg, 1989, which reflect the corresponding knowledge of a person skilled in the art.

The agent (a) is packaged in solid form or as a powder and is therefore substantially anhydrous. Furthermore, the agent (a) is also free from solvent or contains solvent only in very small quantities.

The agent (b) is an aqueous cosmetic carrier. Here, it can be a suitable aqueous or aqueous-alcoholic carrier, for example. For the purpose of the reductive decolorization, such carriers can be creams, emulsions, gels or also surfactant-containing foaming solutions for example, such as shampoos, foam aerosols, foam formulations or other preparations suitable for use on the hair. The agents for reductive removal of color from keratinic fibers are particularly preferably creams or emulsions.

Agent (a) in Container (A)

The multi-component packaging unit (kit-of-parts) according to the invention comprises a first separately packaged container (A) comprising a solid or powdered cosmetic agent (a).

Within the scope of the present invention a solid agent is understood to mean an agent (a) which has a melting point of at least 25° C.; the agent (a) is thus solid at room temperature.

Within the scope of the present invention a solid agent is understood to mean in particular an agent which is present as a shaped article, for example in the form of a tablet or one or more pellets.

In order to produce this shaped article, the agent (a) (or the meltable parts of the agent (a)) can be heated above the melting point, poured into a mold, and then cooled. It is also possible to compact a powdered agent (a) so as to form a shaped article.

The agent (a) can also be present in powder form. Here, powders formed from solid constituents having different grain sizes can be used. However, it is usually preferred if the powders have a grain size that is as uniform as possible, in particular so as to facilitate a uniform dispersion or dissolution of the powder in the agent (b).

A powder is a batch consisting of small, solid particles. The powdered agent (a) preferably has an average particle diameter of at least 20 μm and a BET surface area of from 40 to 400 m²/g (determined in accordance with DIN 66131 with nitrogen).

If the agent (a) is present in powder form, the powder or the powder mixture thus also has a melting point of at least 25° C.

A feature of the solid or powdered agent (a) essential to the invention is its content of one or more sulfinic acid derivatives (a1) from the group consisting of
(H2N)(NH)C(SO₂H) formamidine sulfinic acid,
HN(CH₂SO₂Na)₂disodium[(sulfinatomethyl)amino]methane sulfinate
HN(CH₂SO₂K)₂, dipotassium[(sulfinatomethyl)amino]methane sulfinate
HN(CH₂SO₂H)₂, 1[(sulfinomethyl)amino]methanesulfinic acid
N(CH₂SO₂Na)₃, trisodium[bis(sulfinatomethyl)amino]methane sulfinate
N(CH₂SO₂K)₃, tripotassium[bis(sulfinatomethyl)amino]methane sulfinate
N(CH₂SO₂H)₃, [bis(sulfinomethyl)amino]methanesulfinic acid
H₂NCH(CH₃)SO₂Na, sodium1-aminoethan-1-sulfinate
H₂NCH(CH₃)SO₂K, potassium 1-aminoethan1-sulfinate
H₂NCH(CH₃)SO₂H, 1-aminoethan-1-sulfinic acid,
HN(CH(CH₃)SO₂Na)₂, disodium 1-[(1-sulfinatoethyl)amino]ethan-1-sulfinate
HN(CH(CH₃)SO₂K)₂, dipotassium 1-[(1-sulfinatoethyl)amino]ethan-1-sulfinate
HN(CH(CH₃)SO₂H)₂, 1-[(1-sulfinoethyl)amino]ethan-1-sulfinic acid
N(CH(CH₃)SO2Na)₃, trisodium 1-[bis(1-sulfinatoethyl)amino]ethan-1-sulfinate
N(CH(CH₃)SO₂K)₃, tripotassium 1-[bis(1-sulfinatoethyl)amino]ethan-1-sulinate
and/or
N(CH(CH₃)SO₂H)₃, 1-[bis(1-sulfinoethyl)amino]ethan-1-sulfinic acid.

Formamidine sulfinic acid is alternatively also referred to as thiourea dioxide or as aminoiminomethanesulfinic acid. Formamidine sulfinic acid has the structure of formula (I), but can also be present in the form of its tautomers. Formamidine sulfinic acid has a melting point of 100° C., has the CAS number 1758-73-2, and is commercially obtainable from various providers, for example from Sigma Aldrich.

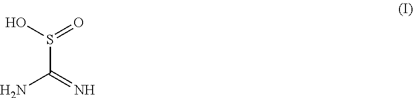

Disodium[(sulfinatomethyl)amino]methane sulfinate is the disodium salt of [(sulfinomethyl)amino]methanesulfinic acid and has the structure of formula (II).

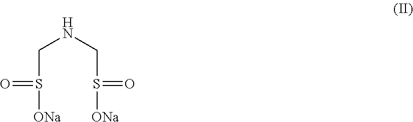

Dipotassium [(sulfinatomethyl)amino]methane sulfinate is the dipotassium salt of [(sulfinomethyl)amino]methanesulfinic acid and has the structure of formula (III).

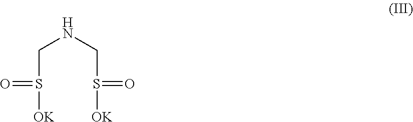

[(Sulfinomethyl)amino]methanesulfinic acid has the structure of formula (IV).

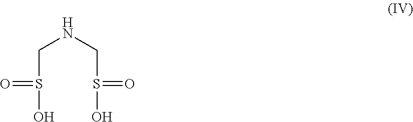

Trisodium[bis(sulfinatomethyl)amino]methane sulfinate is the trisodium salt of [bis(sulfinomethyl)amino]methanesulfinic acid and has the structure of formula (V).

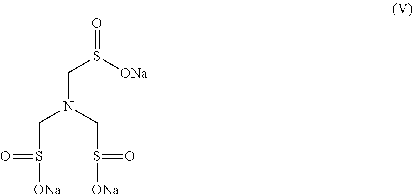

Tripotassium[bis(sulfinatomethyl)amino]methane sulfinate is the tripotassium salt of (sulfinomethyl)amino]methanesulfinic acid and has the structure of formula (VI).

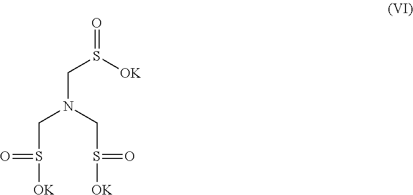

[Bis(sulfinomethyl)amino]methanesulfinic acid has the structure of formula (VII).

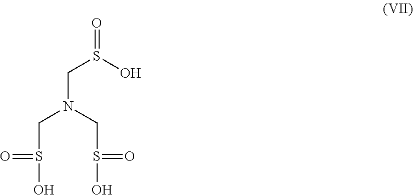 [SMILES: O=S(O)CN(CS(=O)O)CS(=O)O]

Sodium 1-aminoethan-1-sulfinate is the sodium salt of 1-aminoethan-1-sulfinic acid and has the structure of formula (VIII).

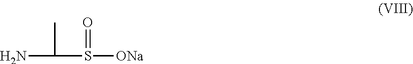

Potassium 1-aminoethan-1-sulfinate is the potassium salt of 1-aminoethan-1-sulfinic acid and has the structure of formula (IX).

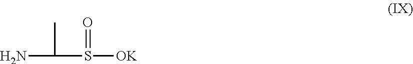

1-aminoethan-1-sulfinic acid has the structure of formula (X).

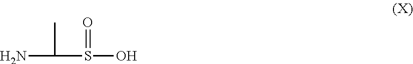

Disodium 1-[(1-sulfinatoethyl)amino]ethan-1-sulfinate is the disodium salt of 1-[(1-sulfinoethyl)amino]ethan-1-sulfinic acid and has the structure of formula (XI).

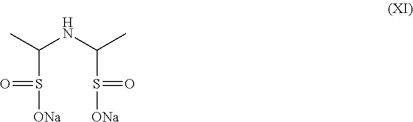

Dipotassium 1-[(1-sulfinatoethyl)amino]ethan-1-sulfinate is the dipotassium salt of 1-[(1-sulfinoethyl)amino]ethan-1-sulfinic acid and has the structure of formula (XII).

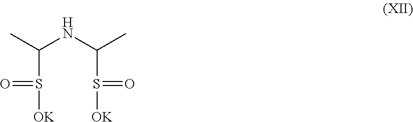

1-[(1-sulfinoethyl)amino]ethan-1-sulfinic acid has the structure of formula (XIII).

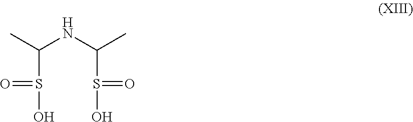

Trisodium 1-[bis(1-sulfinatoethyl)amino]ethan-1-sulfinate is the trisodium salt of 1-[bis(1-sulfinoethyl)amino]ethan-1-sulfinic acid and has the structure of formula (XIV)

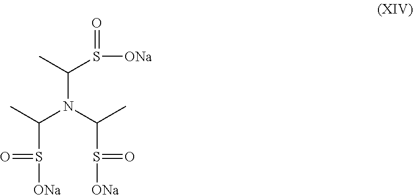

Tripotassium 1-[bis(1-sulfinatoethyl)amino]ethan-1-sulinate is the tripotassium salt of 1-[bis(1-sulfinoethyl)amino]ethan-1-sulfinic acid and has the structure of formula (XV).

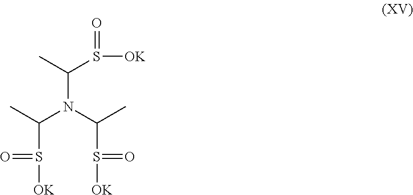

1-[bis(1-sulfinoethyl)amino]ethan-1-sulfinic acid has the structure of formula (XVI).

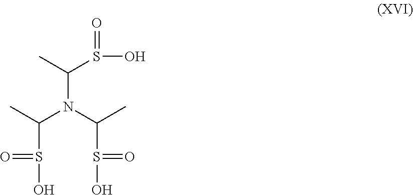 [SMILES: CC(N(C(C)S(=O)O)C(C)S(=O)O)S(=O)O]

The production of the compounds of formulas (II) to (XVI) is described by way of example in EP 0914516 B 1.

The development of the odor of the kit-of-parts according to the invention is co-determined both by the sulfinic acids (a1) contained in the agent (a) and by the further ingredients present in the aqueous cosmetic carrier (b).

A very particularly good decolorization effect with simultaneous particularly low odor development could be obtained with formamidine sulfinic acid, and therefore the use of formamidine sulfinic acid in the solid/powdered agents (a) is very particularly preferred.

A particularly preferred multi-component packaging unit (kit-of-parts) is characterized in that the agent (a) in container (A)
(a1) contains (H2N)(NH)C(SO₂H) formamidine sulfinic acid as sulfinic acid derivative.

The sulfinic acids(s) from group (a1) is/are preferably used in the agent (a) in certain quantity ranges. In order to obtain an optimal decolorization effect, it is preferred when the decolorizing agent contains the sulfinic acid derivative(s) (a1) in a total quantity of from 60.0 to 100.0% by weight, preferably from 70.0 to 100.0% by weight, more preferably from 80.0 to 100.0% by weight, and particularly preferably from 90.0 to 100.0% by weight. Here, all specified quantities relate to the total weight of all sulfinic acid derivatives from the group (a1) contained in the agent (a), and this is set in relation to the total weight of the agent (a).

A particularly preferred multi-component packaging unit (kit-of-parts) is also characterized in that the agent (a) in container (A) contains—in relation to the total weight of the agent (a)—one or more sulfinic acid derivatives from group (a1) in a total quantity of from 60.0 to 100.0% by weight, preferably from 70.0 to 100.0% by weight, more preferably from 80.0 to 100.0% by weight, and particularly preferably from 90.0 to 100.0% by weight.

A further particularly preferred multi-component packaging unit (kit-of-parts) is characterized in that the agent (a) in container (A) contains—in relation to the total weight of the agent (a)—60.0 to 100.0% by weight of formamidine sulfinic acid (a1).

A further particularly preferred multi-component packaging unit (kit-of-parts) is characterized in that the agent (a) in container (A) contains—in relation to the total weight of the agent (a)—70.0 to 100.0% by weight of formamidine sulfinic acid (a1).

A further particularly preferred multi-component packaging unit (kit-of-parts) is characterized in that the agent (a) in container (A) contains—in relation to the total weight of the agent (a)—80.0 to 100.0% by weight of formamidine sulfinic acid (a1).

A further particularly preferred multi-component packaging unit (kit-of-parts) is characterized in that the agent (a) in container (A) contains—in relation to the total weight of the agent (a)—90.0 to 100.0% by weight of formamidine sulfinic acid (a1).

The user wishing to decolorize their dyed hair again applies the reductive decolorizing agent to hair that has previously already been subjected to a hair dyeing process. In the case of an oxidative colorant, the hair has already been oxidatively damaged prior to the decolorization, and the reductive decolorization process may damage the hair further. A central objective is therefore that of attaining the most complete decolorization possible whilst causing the least damage possible to the keratin fibers.

It is known from the prior art that the use of metal salts may potentially have a positive effect on the decolorization effect, but is also associated with the disadvantage of a disproportionately high level of damage to the hair. Keratin fibers which have been decolorized with use of a metal salt became extremely brittle and fragile. Furthermore, with use of metal salts, interactions with the reductively modified dyes have been observed, i.e. color shifts and sometimes an accelerated darkening have been observed. For this reason it is essential to the invention that both the agent (a) and the agent (b) are free from divalent and trivalent metal salts.

For this reason the total content of all metal salts contained in the agent (a) from the group of magnesium salts, zinc salts, iron salts, copper salts and calcium salts lies at a value below 0.1% by weight. The specified weight of 0.1% by weight relates here to the total quantity of all metal salts contained in the agent (a) from the group of magnesium salts, zinc salts, iron salts, copper salts, and calcium salts, and this is set in relation to the total weight of the agent (a).

In the agent (b) as well, the total quantity of all metal salts from the group of magnesium salts, zinc salts, iron salts, copper salts and calcium salts lies at a value below 0.1% by weight. The specified weight of 0.1% by weight relates here again to the total quantity of all metal salts contained in the agent (b) from the group of magnesium salts, zinc salts, iron salts, copper salts, and calcium salts, and this is set in relation to the total weight of the agent (b).

Magnesium salts are, for example, MgCl₂, MgBr_{2,} Mg(OH)₂, Mg(CO₃), Mg(OAc)₂.

Zinc salts are, for example, ZnCl₂, ZnBr_{2,} Zn(OH)₂, Zn(OAc)₂.

Iron salts are, for example, FeCl₂, FeBr₂, Fe(OH)₂, Fe(CO₃), FeCl₃, FeBr₃, Fe(OH)₃, Fe₂(CO₃)₂.

Copper salts are, for example, CuCl, CuBr, Cu(OH), CuCl_{2,} CuBr_{2,} Cu(OH)₂, Cu(OAc)₂.

Calcium salts are, for example, CaCl_{2,} CaBr_{2,} Ca(OH)₂, Ca(CO₃), Ca(OAc)₂.

Other divalent and trivalent metal salts are preferably also not contained in the agents (a) and (b).

In a further preferred embodiment, a kit-of-parts according to the invention is therefore characterized in that
the agent (a) is substantially free from divalent and trivalent metal salts and
the agent (b) is substantially free from divalent and trivalent metal salts.

Bivalent and trivalent metal salts within the sense of the present invention are understood to mean all divalent and trivalent salts of metals from sub-groups 3 to 12 and main groups 3 and 4 —these being in particular:
salts of metals of the 4^{th} sub-group: Ti, Zr, Hf
salts of metals of the 5^{th} sub-group: V, Nb, Ta
salts of metals of the 6^{th} sub-group: Cr, Mo, W
salts of metals of the 7^{th} sub-group: Mn
salts of metals of the 8^{th} sub-group: Fe, Ru, Os
salts of metals of the 9^{th} sub-group: Co, Rh, Ir
salts of metals of the 10^{th} sub-group: Ni, Pd, Pt
salts of metals of the 11^{th} sub-group: Cu, Ag, Au
salts of metals of the 12^{th} sub-group: Zn
salts of metals of the 3^{rd} main group: Al, Ga, In, Tl
salts of metals of the 4^{th} main group: Ge, Sn, Pb (silicon compounds are not considered to be metal salts within the sense of the invention)
salts of metals of the 5^{th} main group: As, Sb, Bi.

Divalent metal salts are understood to be the salts of a twice positively charged metal cation. Trivalent metal salts are understood to be the salts of a thrice positively charged metal cation. In particular, the presence of TiO_{2,} MnCl₂, MnCl_{3,} Mn(OH)₂, Mn(OH)₃, AlCl₃, Al(OH)₃ has proven to be disadvantageous in this context.

The term “substantially free from” is understood to mean that, although certain raw materials in some circumstances could contain metal salts as by-products in small quantities, the deliberate addition of the metal salts to the agents (a) and (b) is avoided.

The total quantity of all metal salts contained in the agent (a) from the group of magnesium salts, zinc salts, iron salts, copper salts and calcium salts lies particularly preferably—in relation to the total weight of the agent (a)—at a value below 0.01% by weight.

The total quantity of all metal salts contained in the agent (b) from the group of magnesium salts, zinc salts, iron salts, copper salts and calcium salts also lies particularly preferably—in relation to the total weight of the agent (b)—at a value below 0.01% by weight.

A particularly preferred multi-component packaging unit (kit-of-parts) is also characterized in that
the total quantity of all metal salts contained in the agent (a) from the group of magnesium salts, zinc salts, iron salts, copper salts and calcium salts lies particularly preferably—in relation to the total weight of the agent (a)—at a value below 0.01% by weight, and
the total quantity of all metal salts contained in the agent (b) from the group of magnesium salts, zinc salts, iron salts, copper salts and calcium salts lies particularly preferably—in relation to the total weight of the agent (b)—at a value below 0.01% by weight.

When mixing the solid or powdered agent (a) with the aqueous carrier agent (b), a rapid and clump-free dissolution of the agent (a) is advantageous in order to attain a uniform decolorization result. For this reason, the agent (a) according to the invention can preferably contain at least one dissolution accelerator.

A particularly preferred multi-component packaging unit (kit-of-parts) is also characterized in that the agent (a) in container (A) additionally contains
(a2) one or more dissolution accelerators.

The term ‘dissolution accelerator’ includes gas-developing components, pre-formed and enclosed gases, disintegrants, and mixtures thereof.

In a preferred embodiment of the present invention, gas-developing components are used as dissolution accelerators. These components react with one another upon contact with water with in-situ formation of gases, which generate a pressure in the tablet, which causes the tablet to disintegrate into smaller particles. One example of a system of this type is constituted by specific combinations of suitable acids with bases. Monovalent, divalent, or trivalent acids having a pKₐ value of from 1.0 to 6.9 are preferred. Preferred acids are citric acid, malic acid, maleic acid, malonic acid, itaconic acid, tartaric acid, oxalic acid, glutaric acid, glutaminic acid, lactic acid, fumaric acid, glycolic acid, and mixtures thereof. Citric acid is particularly preferred. It may be very particularly preferred to use citric acid in particle form, wherein the particles have a diameter smaller than 1000 μm, in particular smaller than 700 μm, very particularly preferably smaller than 400 μm. Further alternative suitable acids are the homopolymers or copolymers of acrylic acid, maleic acid, methacrylic acid or itaconic acid having a molecular weight of from 2,000 to 200,000. Homopolymers of acrylic acid and copolymers of acrylic acid and maleic acid are particularly preferred. Preferred bases in accordance with the invention are alkali metal silicates, carbonates, hydrogen carbonates, and mixtures thereof. Metasilicates, hydrogen carbonates, and carbonates are particularly preferred. Hydrogen carbonates are very particularly preferred. Particulate hydrogen carbonates having a particle diameter of less than 1000 μm, in particular less than 700 μm, very particularly preferably less than 400 μm are particularly preferred. Sodium or potassium salts of the above-mentioned bases are particularly preferred. These gas-developing components are contained in the agent (a) according to the invention preferably in a quantity of at least 10% by weight, in particular of at least 20% by weight.

In a further preferred embodiment of the present invention the gas is pre-formed or enclosed in the agent (a) so that the gas development begins with the onset of the dissolution of the shaped article and accelerates the further dissolution. Examples of suitable gases are air, carbon dioxide, N₂O, oxygen and/or further non-toxic, non-combustible gases.

In a particularly preferred embodiment of the present invention disintegration aids, or what are known as shaped article disintegrants, are incorporated into the shaped article as dissolution accelerators in order to shorten the disintegration times. Shaped article disintegrants or disintegration accelerators are understood in accordance with Römpp (9^{th} edition, volume 6, page 4440) and Voigt “Lehrbuch der pharmazeutischen Technologie” (“Textbook on Pharmaceutical Technology”) (6^{th} edition, 1987, pages 182-184) to be aids which ensure the rapid disintegration of shaped articles in water or gastric juices and also the release of the pharmaceuticals in absorbable form.

These substances, which on account of their effect are also referred to as “disintegrants” enlarge their volume (swell) upon contact with water. Swelling disintegration aids are, for example, synthetic polymers such as polyvinylpyrrolidone (PVP) or natural polymers or modified natural substances such as cellulose and starch and derivatives thereof, alginate, or casein derivatives.

Cellulose-based disintegration agents are used as preferred disintegration agents within the scope of the present invention, and therefore preferred shaped articles contain a cellulose-based disintegration agent of this type in quantities of from 0.5 to 50% by weight, preferably 3 to 30% by weight, in relation to the total shaped article. Pure cellulose has the formal net composition (C₆H₁₀O₅)ₙ and, considered formally, constitutes a β-1,4-polyacetal of cellobiose, which in turn is constructed from two glucose molecules. Suitable celluloses consist here of approximately 500 to 5,000 glucose units and consequently have average molar masses of from 50,000 to 500,000. Within the scope of the present invention, cellulose derivatives which are obtainable by polymer analogous reactions from cellulose can also be used as cellulose-based disintegration agents. Chemically modified celluloses of this type for example comprise products from esterifications or etherifications in which hydroxyl hydrogen atoms have been substituted. However, celluloses in which the hydroxyl groups have been replaced for functional groups which are not bound via an oxygen atom can also be used as cellulose derivatives. The group of cellulose derivatives includes, for example, alkali celluloses, carboxymethyl cellulose (CMC), cellulose esters and ethers, and also amino celluloses. The specified cellulose derivatives are preferably not used as sole cellulose-based disintegration agent, but are used in mixture with cellulose. The content of cellulose derivatives in these mixtures is preferably less than 50% by weight, particularly preferably less than 20% by weight, in relation to the cellulose-based disintegration agent. Pure cellulose, which is free from cellulose derivatives, is particularly preferably used as cellulose-based disintegration agent.

The cellulose used as disintegration aid cannot be used in accordance with the invention in fine-particle form, but is converted into a coarser form, for example is granulated or compacted, prior to being admixed to the pre-mixtures that are to be pressed. The particle sizes of such disintegration agents are usually above 200 μm, preferably at least 90% by weight between 300 and 1600 μm, and in particular at least 90% by weight between 400 and 1200 μm. The disintegration aids according to the invention are commercially obtainable for example under the name Arbocel® from the company Rettenmaier. A preferred disintegration aid is, for example, Arbocel® TF-30-HG.

Microcrystalline cellulose is used as a preferred cellulose-based disintegration agent or as a constituent of this component. This microcrystalline cellulose is obtained by partial hydrolysis of celluloses under conditions which act only on the amorphous regions (approximately 30% of the total cellulose mass) of the celluloses and completely dissolve these, however the crystalline regions (approximately 70%) remain unaffected. A subsequent disaggregation of the micro-fine celluloses produced by the hydrolysis provides the microcrystalline celluloses, which have primary particle sizes of approximately 5 μm and for example can be compacted to form granulates having an average particle size of 200 μm. Suitable microcrystalline cellulose is commercially obtainable for example under the trade name Avicel®.

The accelerated dissolution of the solid agent (a) can also be achieved in accordance with the invention by pre-granulation of the further constituents of the shaped article.

In a preferred embodiment of the shaped articles according to the invention, these contain, in particular besides at least one cellulose-based disintegrant, also a mixture of starch and at least one saccharide for dissolution acceleration. Disaccharides are saccharides used with preference in this embodiment. Said mixture is preferably present with a weight ratio of starch and the used saccharides of from 10:1 to 1:10, particularly preferably of from 1:1 to 1:10, very particularly preferably of from 1:4 to 1:8 in the shaped article.

The used disaccharides are preferably selected from lactose, maltose, sucrose, trehalose, turanose, gentiobiose, melibiose, and cellobiose. Lactose, maltose, and sucrose are particularly preferably used, and lactose is very particularly preferably used in the shaped articles according to the invention.

The starch-saccharide mixture is contained in the shaped article in a quantity of from 5 to 60% by weight, preferably of from 20 to 40% by weight, in relation to the mass of the total shaped article.

A particularly preferred multi-component packaging unit (kit-of-parts) is also characterized in that the agent (a) in the container (A) additionally contains
(a2) one or more dissolution accelerators from the group consisting of homopolymers of (meth)acrylic acid, copolymers of (meth)acrylic acid, polyvinylpyrrolidone (PVP), celluloses, cellulose derivatives, starch, starch derivatives, alginates and/or casein derivatives.

Furthermore, the agent (a)—in particular when present packaged in powder form—can preferably contain one or more anticaking agents. An example of a suitable anticaking agent is particulate silicon dioxide. A clumping of the powder caused by moisture can be prevented or minimized by the use of the anticaking agent.

It is particularly preferred when the particulate silicon dioxide has an average particle diameter of at least 40 μm, very particularly preferably of at least 100 μm. It is preferred when the particulate silicon dioxide has at most an average particle diameter of 500 μm.

It is also advantageous in accordance with the invention when the particulate silicon dioxide has pores, in particular mesopores and/or macropores. The volume of these pores preferably takes up 10 to 80% of the volume of a silicon dioxide particle. It is particularly preferred when the sum of the volume of those pores having a diameter of less than 5 nm accounts for no more than 5% of the total pore volume.

It is preferred when the particulate silicon dioxide has a pore volume of from 0.5 to 3.0 mL/g.

Examples of the silicon dioxide that can be used in accordance with the invention are described in the two documents WO 03/037287 and EP 725037 A1, to which reference is made here expressly and fully.

It is particularly preferred to adapt the quantity of used particulate silicon dioxide to the quantity of sulfinic acids (a1) contained in the agent (a).

Preferably, 0.5 to 4.0% by weight of the particulate silicon dioxide, in relation to the weight of the agent (a), is used in the agents (a).

Particulate silicon dioxide that can be used in accordance with the invention is sold for example under the trade name Aerosil 200 by the company Evonik.

A particularly preferred multi-component packaging unit (kit-of-parts) is also characterized in that the agent (a) in container (A) additionally contains
(a3) particulate silicon dioxide as anticaking agent.

A further particularly preferred multi-component packaging unit (kit-of-parts) is also characterized in that the agent (a) in container (A) additionally contains,
(a3) as anticaking agent, particulate silicon dioxide having an average particle diameter of at least 20 μm and a BET surface of from 40 to 400 m²/g (determined in accordance with DIN 66131 with nitrogen).

As previously described, an agent (a) according to the invention contains at least one sulfinic acid derivative from the group (a1). The sulfinic acid derivatives from this group are characterized by a minimal development of odor during their use. With regard to an optimal solution of the problem addressed by the invention, it has also proven to be particularly advantageous to dispense with the use of other reducing agents that have an intense odor. For this reason, it is particularly preferred when the agent (a) is substantially free from further reducing agents from the group consisting of sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, and thioglycolic acid.

A particularly preferred multi-component packaging unit (kit-of-parts) is also characterized in that—in relation to the total weight of the agent (a)—
the total content of all reducing agents contained in the agent (a) from the group consisting of sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, thioglycolic acid and all physiologically acceptable salts of these compounds lies at a value below 0.5% by weight, preferably below 0.1% by weight, and particularly preferably below 0.05% by weight.

The specified quantity values relate here to the total weight of all compounds contained in the agent (a) from the group formed from sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, thioglycolic acid and all physiologically acceptable salts of these compounds, and this is set in relation to the total weight of the agent (a).

Agent (b) in Container (B)

The multi-component packaging unit according to the invention comprises a second separately packaged container (B), which contains an aqueous agent (b). This agent (b) is a cosmetic carrier formulation which can be aqueous or aqueous-alcoholic.

The agent (b) preferably has a water content of at least 60.0% by weight, preferably of at least 70.0% by weight, more preferably of at least 80.0% by weight, and very particularly preferably of at least 90.0% by weight.

A particularly preferred multi-component packaging unit (kit-of-parts) is also characterized in that the agent (b) in container (B)—in relation to the total weight of the agent (b)—has
(b 1) a water content of at least 60.0% by weight, preferably of at least 70.0% by weight, more preferably of at least 80.0% by weight, and very particularly preferably of at least 90.0% by weight.

Various reducing agents pass through their point of optimum efficacy within a specific pH value range. Ready-to-use decolorizing agents comprising the sulfinic acid derivatives of group (a1) contain their best effect in the alkaline range.

The ready-to-use decolorizing agent is—as already described previously—produced shortly before application by mixing the agents (a) and (b). The ready-to-use decolorizing agent therefore preferably contains at least one alkalising agent. It is preferable, for stability and storage reasons, to incorporate the alkalising agent(s) into the cosmetic carrier agent (b).

A quick dissolution of the agent (a) and a very good decolorization effect could be observed when the agent (b) in container (B) has been set to a pH in the range of from 7.5 to 12.0, preferably from 8.0 to 11.5, more preferably from 8.5 to 11.0, and particularly preferably from 9.0 to 10.5.

A particularly preferred multi-component packaging unit (kit-of-parts is also characterized in that the agent (b) in container (B) has a pH value which lies in the range of from 7.5 to 12.0, preferably from 8.0 to 11.5, more preferably from 8.5 to 11.0, and particularly preferably from 9.0 to 10.5.

All pH values of the present invention were measured using a glass electrode of the N 61 type from the company Schott at a temperature of 22° C.

By way of example, basic amino acids such as arginine, lysine, ornithine and/or histidine can be used as alkalising agent in the cosmetic carrier agent (b).

Further suitable alkalising agents are inorganic alkalising agents from the group composed of sodium hydroxide, potassium hydroxide, sodium carbonate, sodium hydrogen carbonate, potassium carbonate and/or potassium hydrogen carbonate.

Nitrogen-containing alkalising agents from the group of alkanolamines have proven to be suitable in this context, Particularly preferred alkanolamines can be selected for example from the group composed of 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1, 1-aminopentan-3-ol, 1-aminopentan-4-ol, 3-amino-2-methylpropan-1-ol, 1-amino-2-methylpropan-2-ol, 3-aminopropan-1,2-diol and/or 2-amino-2-methylpropan-1,3-diol.

In principle, ammonia can also be used as alkalising agent. In order to avoid any development of odor however, it is particularly advantageous to also dispense with the use of alkalising agents that have an intense odor, such as ammonia.

A particularly preferred multi-component packaging unit (kit-of-parts) is also characterized in that the agent (b) additionally contains at least one alkalising agent from the group composed of monoethanolamine, triethanolamine, 2-amino-2-methylpropanol, arginine, lysine and/or histidine.

With regard to an optimal solution to the problem addressed by the invention, it has proven to be particularly advantageous to dispense with the use of other reducing agents that have an intense odor. For this reason, it is also particularly preferred when the agent (b) is also substantially free from further reducing agents from the group composed of sodium sulphite, sodium hydrogen sulphite, potassium sulphite, potassium hydrogen sulphite, ammonium sulphite, hydroxyl methanesulfinic acid, aminomethanesulfinic acid, cysteine, thiolactic acid, and thioglycolic acid.

A particularly preferred multi-component packaging unit (kit-of-parts) is also characterized in that—in relation to the total weight of the agent (b)—
the total content of all reducing agents contained in the agent (b) from the group composed of sodium sulphite, sodium hydrogen sulphite, potassium sulphite, potassium hydrogen sulphite, ammonium sulphite, hydroxyl methanesulfinic acid, aminomethanesulfinic acid, cysteine, thiolactic acid, thioglycolic acid and all physiologically acceptable salts of these compounds lies at a value below 0.5% by weight, preferably below 0.1% by weight, and particularly preferably below 0.05% by weight.

The specified quantity values relate here to the total weight of all compounds contained in the agent (b) from the group formed from sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, thioglycolic acid and all physiologically acceptable salts of these compounds, and this is set in relation to the total weight of the agent (b).

The agent (b) is provided as a liquid preparation, to which further surface-active substances can be added. These are preferably selected from anionic, zwitterionic, amphoteric and non-ionic surfactants and emulsifiers.

As anionic surfactants, the agent (b) can contain, for example, fatty acids, alkyl sulfates, alkyl ether sulfates, and ether carboxylic acids having 10 to 20 C atoms in the alkyl group and up to 16 glycol ether groups in the molecule.

The agent (b) can also contain one or more zwitterionic surfactants, such as betaines, N-alkyl-N,N-dimethylammonium glycinates, N-acyl-aminopropyl-N,N-dimethylammonium glycinates, and 2-alkyl-3-carboxymethyl-3-hydroxyethyl imidazolines.

Agents (b) that are suitable in accordance with the invention are also characterized in that the agent (b) additionally contains at least one amphoteric surfactant. Preferred amphoteric surfactants are N-alkyl glycines, N-alkyl propionic acids, N-alkyl amino butyric acids, N-alkyl imino dipropionic acids, N-hydroxyethyl-N-alkyl amido propyl glycines, N-alkyl taurines, N-alkyl sarcosines, 2-alkyl amino propionic acids, and alkyl amino acetic acids. Particularly preferred amphoteric acids are N-coco alkyl amino propionate, coco acylaminoethylamino propionate, and C₁₂-C₁₈ acylsarcosine.

It has also proven to be advantageous when the agent (b) contains further, non-ionogenic surface-active substances. Preferred non-ionic surfactants have proven to be alkyl polyglycosides and alkylene oxide addition products with fatty alcohols, fatty acids, and fatty acid glycerides with, in each case, 2 to 50 mol of ethylene oxide per mol of fatty alcohol or fatty acid. Preparations that have excellent properties are likewise obtained when they contain fatty acid esters of ethoxylated glycerol as non-ionic surfactants. It is very particularly preferred when the agent (b), as non-ionic surfactant, contains an ethoxylated castor oil with, in each case, 2 to 50 mol of ethylene oxide per mol of fatty acid or an ethoxylated hydrogenated castor oil with, in each case, 2 to 50 mol of ethylene oxide per mol of fatty acid. The use of PEG-40 castor oil is particularly preferred in this context.

The non-ionic, zwitterionic, or amphoteric surfactants are used in proportions of from 0.1 to 15.0% by weight, preferably 0.5 to 10.0% by weight, and very particularly preferably from 0.7 to 5.0% by weight, in relation to the total amount of the agent.

It is very particularly preferred when the agent (b) contains at least one anionic surfactant.

A particularly preferred multi-component packaging unit (kit-of-parts) is also characterized in that the agent (b) in container (B) additionally contains at least one anionic surfactant.

A particularly preferred multi-component packaging unit (kit-of-parts) is also characterized in that the agent (b) in container (B) additionally contains at least one anionic surfactant from the group C₈-C₃₀ fatty acids, C₈-C₃₀ alkyl sulfates, C₈-C₃₀ alkyl ether sulfates, and ether carboxylic acids having 10 to 20 C atoms in the alkyl group and up to 16 glycol ether groups in the molecule.

The anionic surfactants can be used for example in quantities equalling a total quantity of from 0.2 to 8.0% by weight, preferably from 0.4 to 5.0% by weight, and particularly preferably from 0.6 to 2.5% by weight—in relation to the total weight of the agent (b).

A particularly preferred multi-component packaging unit (kit-of-parts) is also characterized in that the agent (b) in container (B) contains—in relation to the total weight of the agent (b)—one or more anionic surfactants in a total quantity of from 0.2 to 8.0% by weight, preferably from 0.4 to 5.0% by weight, and particularly preferably from 0.6 to 2.5% by weight.

The aqueous carrier agent (b) can be formulated both as a gel and as an emulsion. Within the sense of the invention, an emulsion is understood to mean a finely distributed mixture of two normally immiscible phases without visible segregation. When the agent (b) is formulated as an emulsion, it particularly preferably additionally contains at least one fat constituent.

“Fat constituents” within the sense of the invention are understood to mean organic compounds having a solubility in water at room temperature (22° C.) and atmospheric pressure (760 mmHg) of less than 1% by weight, preferably of less than 0.1% by weight.

The agent (b) very particularly preferably contains, as fat constituent, one or more C₁₂-C₃₀ fatty alcohols. It has surprisingly been found that—when the agent (b) is present as an emulsion and contains at least one C₁₂-C₃₀ fatty alcohol—the development of odor could be minimized further still.

The C₁₂-C₃₀ fatty alcohols can be saturated, mono- or polyunsaturated, linear or branched fatty alcohols having 12 to 30 C atoms.

Examples of preferred linear saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidon alcohol ((5Z, 8Z,11Z,14Z)-eicosa-5, 8,11,14-tetraen-1-ol), erucyl alcohol ((13Z)-docos-13-en-1-ol) and/or brassidyl alcohol ((13E)-docosen-1-01).

A particularly preferred multi-component packaging unit (kit-of-parts) is also characterized in that the agent (b) in container (B) additionally contains at least one C₁₂-C₃₀ fatty alcohol.

The multi-component packaging unit according to the invention is used for the reductive decolorization of dyed keratinic fibers. The agents (a) and (b) here together form the ready-to-use decolorizing agent, which contains a reducing agent. The agents (a) and (b) therefore preferably do not contain any oxidizing agent for reasons of incompatibility and in order to avoid uncontrollable exothermic reactions.

Here, oxidizing agents are understood to mean, in particular, the oxidizing agents that can also be used for oxidative decolorization, such as hydrogen peroxide and persulfates (potassium persulfate (alternatively potassium peroxodisulfate), sodium persulfate (sodium peroxodisulfate), and ammonium persulfate (alternatively ammonium peroxodisulfate)). Thus, neither of the agents (a) or (b) preferably contains the aforementioned oxidizing agents.

In a further preferred embodiment, a multi-component packaging unit (kit-of-parts) according to the invention is therefore characterized in that
the total content of the hydrogen peroxide contained in the agent (b)—in relation to the total weight of the agent (b)—lies at a value below 1.0% by weight, preferably below 0.5% by weight, more preferably below 0.1% by weight, and particularly preferably below 0.01% by weight.

In a further preferred embodiment, a multi-component packaging unit (kit-of-parts) according to the invention is also characterized in that
the total content of the persulfate salts contained in the agent (b)—in relation to the total weight of the agent (b)—lies at a value below 1.0% by weight, preferably below 0.5% by weight, more preferably below 0.1% by weight, and particularly preferably below 0.01% by weight.

Agent (c) in Container (C)

The multi-component packaging unit according to the invention can optionally also comprise a third container (C), which contains the agent (c) packaged separately. The agent (c) can be, for example, a pre-treatment agent or a post-treatment agent.

It is also conceivable to add the agent (c) of the multi-component packaging unit as nourishing drops, wherein the agent (c) for example contains a nourishing or conditioning component which is packaged separately from the agents (a) and (b) for stability reasons. In this case the agents (a), (b) and (c) can be mixed with one another to produce the ready-to-use decolorizing agent.

Further reducing agents having an intense odor are also omitted from this third component (c) where possible.

In a further preferred embodiment a multi-component packaging unit (kit-of-parts) is therefore characterized in that it packages separately from one another
—a container (C) containing a cosmetic agent (c), wherein
the total content of all metal salts contained in the agent (c) from the group of magnesium salts, zinc salts, iron salts, copper salts, and calcium salts—in relation to the total weight of the agent (c)—lies at a value below 0.1% by weight.

The agents (b) and, where applicable, (c) according to the invention can additionally contain at least one oil component. In the case of the agent (b), this oil component which can be used additionally is understood to be a component different from C₁₂-C₃₀ fatty alcohols. Oil components that are suitable in accordance with the invention are in principle all oils and fatty substances and mixtures thereof with solid paraffins and waxes. Oil components of which the solubility in water at 20° C. is less than 1% by weight, in particular less than 0.1% by weight, are preferred. The melting point of the individual oil or fat components lies preferably below approximately 40° C. Oil components which are liquid at room temperature, i.e. below 25° C., can be particularly preferred in accordance with the invention. With use of a number of oil and fat components and optionally solid paraffins and waxes, however, it is generally also sufficient when the mixture of the oil and fat components and optionally paraffins and waxes meets these conditions.

A preferred group of oil components is constituted by vegetable oils. Examples of such oils are apricot kernel oil, avocado oil, sunflower oil, olive oil, soybean oil, rapeseed oil, almond oil, jojoba oil, orange oil, wheat germ oil, peach kernel oil and the liquid components of coconut oil. Other triglyceride oils are also suitable, however, such as the liquid components of beef tallow and synthetic triglyceride oils.

A further particularly preferred group of oil components that can be used in accordance with the invention is constituted by liquid paraffin oils and synthetic hydrocarbons and also di-n-alkylethers having a total of between 12 and 36 C atoms, in particular 12 to 24 C atoms, such as di-n-octylethers, di-n-decylethers, di-n-nonylethers, di-n-undecylethers, di-n-dodecylethers, n-hexyl-n-octylethers, n-octyl-n-decylethers, n-decyl-n-undecylethers, n-undecyl-n-dodecylethers and n-hexyl-n-undecylethers and di-tert-butylethers, di-iso-pentylethers, di-3-ethyldecylethers, tert.-butyl-n-octylethers, iso-pentyl-n-octylethers and 2-methyl-pentyl-n-octylethers. The compounds 1,3-di-(2-ethyl-hexyl)-cyclohexane (Cetiol® S) and di-n-octylether (Cetiol® OE), which are obtainable as commercial products, can be preferred.

Oil components that can also be used in accordance with the invention are fatty acid and fatty alcohol esters. The monoesters of fatty acids with alcohols having 3 to 24 C atoms are preferred. This substance group is constituted by the products of esterification of fatty acids having 8 to 24 C atoms, such as caproic acid, caprylic acid, 2-ethylhexanoic acid, capric acid, lauric acid, isotridecanoic acid, myristic acid, palmitic add, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, which occur for example in the event of the pressure-splitting of natural fats and oils, in the event of reduction of aldehydes from Roelens oxosynthesis, or the dimerisation of unsaturated fatty acids, with alcohols such as isopropyl alcohol, glycerol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachyl alcohol, gadoleyl alcohol, behenyl alcohol, erucyl alcohol and brassidyl alcohol and technical mixtures thereof, which occur for example in the event of high-pressure hydrogenation of technical-grade methyl esters based on fats and oils or aldehydes from Roelens oxosynthesis, and also as monomer fraction in the event of the dimerisation of unsaturated fatty alcohols. Isopropyl myristate, isononanoic acid C₁₆C₁₈ alkyl ester (Cetiol® SN), stearic acid 2-ethylhexyl ester (Cetiol® 868), cetyl oleate, glycerol tricaprylate, coconut fatty alcohol caprinate/caprylate and n-butyl stearate are particularly preferred in accordance with the invention.

Dicarboxylic acid esters such as di-n-butyl adipate, di-(2-ethylhexyl) adipate, di-(2-ethylhexyl) succinate and di-isotridecyl acelaat, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, and neopentyl glycol dicaprylate are also oil components that can be used in accordance with the invention, as well as complex esters such as diacetyl glycerol monostearate.

Oil components that can be used with preference in accordance with the invention are, lastly, also silicone oils, in particular dialkyl and alkylaryl siloxanes, such as dimethyl polysiloxane and methylphenyl polysiloxane and alkoxylated and quaternized analogues thereof and cyclic siloxanes. Examples of such silicones are the products marketed by Dow Corning under the names of DC 190, DC 200 and DC 1401 and the commercial products DC 344 and DC 345 of Dow Corning, Q2-7224 (manufacturer: Dow Corning; a stabilized trimethyl silyl amodimethicone), Dow Corning® 929 emulsion (containing a hydroxyl amino-modified silicone which is also known as Amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, Quaternium-80). Silicone oils having a kinematic viscosity up to 50,000 cSt measured at 25° C. can be preferred in the context of the invention. Silicone oils having kinematic viscosities up to 10,000 cSt measured at 25° C. are very particularly preferred. The viscosities are determined here by the ball drop method in accordance with the “British Standard 188” method. Comparable values are obtained using manufacturer's test procedures similar to the “British Standard 188” method, for example the “CTM 0577” test procedure of the Dow Corning Corporation.

In a particular embodiment, cyclic siloxanes are used in particular as oil components, such as the products Dow Corning® 344, Dow Corning® 345, Dow Corning® 244, Dow Corning® 245 or Dow Corning® 246 having kinematic viscosities of up to 10,000 cSt determined at 25° C. in accordance with the manufacturer's instructions.

Oil components that can be used in accordance with the invention are lastly also dialkyl carbonates, as described in detail in DE OS19710154, to which reference is made expressly. Dioctyl carbonates, in particular the di-2-ethylhexyl carbonate, are preferred oil components within the scope of the present invention.

In a further embodiment emulsifiers can be used in the agents (a), (b) and/or (c) according to the invention. Emulsifiers that are solid at room temperature can be used in the agent (a) in accordance with the invention. Emulsifiers cause the formation of water- or oil-stable adsorption layers at the phase boundary, which layers protect the dispersed drops against coalescence and thus stabilize the emulsion. Emulsifiers, similarly to surfactants, are therefore constructed from a hydrophobic and a hydrophilic molecule part. Hydrophilic emulsifiers preferably form O/W emulsions, and hydrophobic emulsifiers preferably form W/O emulsions. An emulsion is a droplet-like distribution (dispersion) of one liquid in another liquid with expenditure of energy to create stabilizing phase boundaries by means of surfactants. The selection of these emulsifying surfactants or emulsifiers is focused here on the substances to be dispersed and the particular outer phase and the fineness of the emulsion. More detailed definitions and properties of emulsifiers can be found in “H.-D.Dörfler, Grenzflächen- und Kolloidchemie, VCH Verlagsgesellschaft mbH. Weinheim, 1994” (Interface and Colloid Chemistry, VCH publishing group mbH). Emulsifiers that can be used in accordance with the invention are, for example,
Addition products of from 4 to 100 mol ethylene oxide and/or 0 to 5 mol propylene oxide with linear fatty alcohols having 8 to 22 C atoms, with fatty acids having 12 to 22 C atoms, and with alkyl phenols having 8 to 15 C atoms in the alkyl group,
C₁₂-C₂₂ fatty acid mono esters or fatty acid diesters of addition products of from 1 to 30 mol ethylene oxide with polyols having 3 to 6 carbon atoms, in particular with glycerol,
Ethylene oxide and polyglycerol addition products with methyl glucoside fatty acid esters, fatty acid alkanolamides and fatty acid glucamides,
C₈-C₂₂ alkyl mono glycosides and alkyl oligo glycosides and ethoxylated analogues thereof, wherein oligomerization degrees of from 1.1 to 5, in particular 1.2 to 2.0, and glucose as sugar component are preferred,
Mixtures of alkyl (oligo) glucosides and fatty alcohols, for example the commercially obtainable product Montanov®68,
Addition products of from 5 to 60 mol ethylene oxide with castor oil and hardened castor oil,
Partial esters of polyols having 3-6 carbon atoms with saturated fatty acids having 8 to 22 C atoms,
Sterols. Sterols are understood to be a group of steroids which carry a hydroxyl group at C atom 3 of the steroid skeleton and which are isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, i.e. what are known as mycosterols.
Phospholipids. These are understood to be, in particular, the glucose phospholipids, which for example are obtained as lecithins or phosphatidylcholines from, for example, egg yolk or plant seeds (for example soybeans).
Fatty acid esters of sugars and sugar alcohols, such as sorbitol.
Polyglycerols and polyglycerol derivatives, such as polyglycerol poly-12-hydroxystearate (commercial product Dehymuls® PGPH),
Linear and branched fatty acids having 8 to 30 C atoms, and Na, K, Ca, Mg and Zn salts thereof.

The agents according to the invention contain the emulsifiers preferably in quantities of from 0.1 to 25% by weight, in particular 0.1 to 3% by weight, based on the particular total composition.

The agents according to the invention can preferably contain at least one non-ionogenic emulsifier having an HLB value of from 8 to 18, in accordance with the definitions detailed in Römpp-Lexikon Chemie (Römpp's Chemistry Lexicon) (Hrg. J. Falbe, M.Regitz), 10th edition, Georg Thieme publishers Stuttgart, New York, (1997), page 1764. Non-ionogenic emulsifiers having an HLB value of 10-15 can be particularly preferred in accordance with the invention.

It has also proven to be advantageous when the agents according to the invention contain a nourishing active substance, selected from protein hydrolysates and derivatives thereof.

Suitable protein hydrolysates are, in particular, elastin, collagen, keratin, milk, albumen, silk protein, soya protein, almond protein, pea protein, potato protein, oat protein, maize protein and wheat protein hydrolysates. Plant-based products can be preferred in accordance with the invention.

Suitable derivatives are, in particular, quaternized protein hydrolysates. Examples of this class of compounds are the products available on the market under the names Lamequat®L (CTFA name: Lauryldimonium Hydroxypropylamino Hydrolyzed Animal Protein; Grünau), Croquat®WKP and Gluadin®WQ. The last-mentioned plant-based product can be preferred.

The protein derivatives are contained in the agents according to the invention preferably in quantities of from 0.1 to 10% by weight, based on the total quantity of the agent. Quantities of from 0.1 to 5% by weight are preferred.

The agents (a), (b) and/or, where applicable, (c) according to the invention preferably also contain at least one conditioning active substance.

Cationic polymers can be considered as preferred conditioning active substances. These are generally polymers which contain a quaternary nitrogen atom, for example in the form of an ammonium group.

Preferred cationic polymers are, for example,
Quaternized cellulose derivatives, as are commercially obtainable under the names Celquat® and Polymer JR®. The compounds Celquat® H 100, Celquat® L 200 and Polymer JR® 400 are preferred quaternized cellulose derivatives.
Polysiloxanes with quaternary groups.
Polymers of dimethyl diallyl ammonium salts and copolymers thereof with esters and amides of acrylic acid and methacrylic acid. The commercially obtainable products under the names Merquat®100 (poly(dimethyl diallyl ammonium chloride)) and Merquat®550 (dimethyl diallyl ammonium chloride actylamide copolymer) are examples of cationic polymers this type.
Copolymers of vinyl pyrrolidone with quaternized derivatives of dialkylamino acrylate and methacrylate, such as vinyl pyrrolidone dimethyl amino methacrylate copolymers quaternized with diethyl sulphate. Such compounds are commercially obtainable under the names Gafquat®734 and Gafquat®755.
Vinyl pyrrolidone-vinyl imidazolinium methochloride copolymers, as are sold under the name Luviquat®.
Quaternized polyvinyl alcohol,
   and the polymers known under the names
Polyquaternium 2,
Polyquaternium 17,
Polyquaternium 18 and
Polyquaternium 27
   with quaternary nitrogen atoms in the main polymer chain.

Amphopolymers are also suitable as conditioning active substances. The encompassing term amphopolymers includes amphoteric polymers, i.e. polymers which contain both free amino groups and free —COOH or SO₃H groups in the molecule and are capable of forming inner salts, zwitterionic polymers which contain quaternary ammonium groups and —COO⁻ or —SO₃⁻ groups, and polymers which contain —COOH or SO₃H groups and quaternary ammonium groups. One example of an amphopolymer that can be used in accordance with the invention is the acrylic resin obtainable under the name Amphomer®, which constitutes a copolymer of tert.-butylaminoethyl methacrylate, N-(1,1,3,3-tetramethylbutyl)acrylamide and two or more monomers from the group composed of acrylic acid, methacrylic acid and simple esters thereof. Amphopolymers that are likewise preferred are composed of unsaturated carboxylic acids (for example acrylic and methacrylic acid), cationically derived unsaturated carboxylic acids (for example acrylamidopropyl trimethylammonium chloride) and optionally further ionic or non-ionogenic monomers, as can be inferred for example from the published, unexamined German application 39 29 973 and the prior art cited there. Terpolymers of acrylic acid, methacrylate and methacrylamidopropyltrimonium chloride, as are commercially obtainable under the name Merquat®2001 N and the commercial product Merquat®280 are amphopolymers that are particularly preferred in accordance with the invention.

The cationic or amphoteric polymers are contained in the preparations according to the invention preferably in quantities of from 0.1 to 5% by weight, based on the total preparation.

Silicone gums, such as the commercial product Fancorsil LIM-1, and anionic silicones, such as the product Dow Corning®1784, are also suitable as conditioning active substances.

Examples of the cationic surfactants that can be used as conditioning active substances in the agents according to the invention are, in particular, quaternary ammonium compounds. Ammonium halides are preferred, in particular chlorides and bromides, such as alkyl trimethyl ammonium chlorides, dialkyl dimethyl ammonium chlorides, and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride. The quaternary ester compounds, which can be biologically degraded very effectively, i.e. what are known as “esterquats”, such as the methyl hydroxyalkyl dialkoyloxyalkyl ammonium methosulphates sold under the trade names Dehyquart® and Stepantex®, can also be used.

Alkylamidoamines, in particular fatty acid amidoamines, such as the stearyl amidopropyl dimethylamine obtainable under the name Tego Amid®S 18, are characterized, besides a good conditioning effect, especially by their good biological degradability.

It may also be preferable to color the individual phases with dyes in order to attain particularly good aesthetics. These dyes are preferably only soluble in the aqueous phase or only in at least a non-aqueous phase in a quantity that leaves a corresponding coloration visible to the observer. It is also possible to color both the non-aqueous and the aqueous phase with different dyes, preferably in different colors. The coloring of just a non-aqueous phase, however, is preferred.

Further conventional constituents for the agents according to the invention are:
Anionic surfactants, such as soaps, alkyl sulphates and alkyl polyglycol ether sulphates, salts of ethercarboxylic acids of formula R—O—(CH₂—CH₂O)x-CH₂—COOH, in which R is a linear alkyl group having 10 to 22 C atoms and x=0 or 1 to 16, acyl sarcosides, acyl taurides, acyl isethionates, sulfosuccinic acid mono and dialkyl esters, linear alkanesulfonates, linear alpha-olefin sulfonates, alpha-sulfo fatty acid methyl esters, and esters of tartaric acid and citric acid, alkyl glycosides or alcohols which constitute addition products of approximately 2-15 molecules of ethylene oxide and/or propylene oxide with fatty alcohols having 8 to 22 C atoms.
Zwitterionic surfactants, such as betaines and 2-alkyl-3-carboxymethyl-3-hydroxyethyl imidazolines.
Ampholytic surfactants, such as N-alkyl glycines, N-alkyl propionic acids, N-alkyl amino butyric acids, N-alkyl imino dipropionic acids, N-hydroxyethyl-N-alkyl amido propyl glycines, N-alkyl taurines, N-alkyl sarcosines, 2-alkyl amino propionic acids, and alkyl amino acetic acids.
Non-ionic surfactants, such as addition products of from 2 to 30 mol ethylene oxide and/or 0 to 5 mol propylene oxide with linear fatty alcohols having 15 to 22 C atoms, with fatty acids having 12 to 22 C atoms, and with alkylphenols having 8 to 15 C atoms in the alkyl group, C₁₂-C₂₂ fatty acid mono and diesters of addition products of from 1 to 30 mol ethylene oxide with glycerol, C₈-C₂₂ alkylmono and oligoglycosides and ethoxylated analogues thereof, and addition products of from 5 to 60 mol ethylene oxide with castor oil and hardened castor oil.
Non-ionic polymers, such as vinyl pyrrolidone/vinyl acrylate copolymers, polyvinyl pyrrolidone and vinyl pyrrolidone/vinyl acetate copolymers.
Anionic polymers, such as polyacrylic and polymethacrylic acids, salts thereof, copolymers thereof with acrylic acid and methacrylic acid esters and amides, and derivatives thereof obtained by cross-linking with polyfunctional agents, polyoxy carboxylic acids, such as polyketo and polyaldehyde carboxylic acids and salts thereof, and polymers and copolymers of crotonic acid with esters and amides of acrylic and methacrylic acid, such as vinyl acetate-crotonic acid and vinyl acetate-vinyl propionate-crotonic acid copolymers,
Organic thickening agents, such as agar-agar, guar gum, alginates, cellulose ethers such as methyl and methyl hydroxyl propyl cellulose, gelatins, pectins, and/or xanthan gum. Ethoxylated fatty alcohols, in particular those with limited homologue distribution, as are available on the market for example as commercial product under the name Arlypon F (Henkel), alkoxylated methylglucoside esters, such as the commercial product Glucamate® DOE 120 (Amerchol), and ethoxylated propylene glycol ester, such as the commercial product Antil® 141 (Goldschmidt), can be preferred organic thickening agents.
Structuring substances such as glucose and maleic acid.
Hair-conditioning compounds such as phospholipids, for example soy lecithin, egg lecithin and cephalin.
Perfume oils.
Solubilizers, such as ethanol, isopropanol, ethylene glycol, propylene glycol, glycerol, diethylene glycol and ethoxylated triglycerides and fatty alcohol ethoxylates and derivatives thereof.
Anti-dandruff active substances, such as climbazole, piroctone, olamine and zinc omadine.
Active substances such as bisabolol, allantoin, panthenol, niacinmide, tocopherol and plant extracts.
Light stabilizers.
Consistency promoters, such as sugar esters, polyol esters or polyol alkyl ethers.
Fatty acid alkanolamides.
Complexing agents, such as EDTA, NTA, β-alanine diacetic acid, and phosphonic acids,
Swelling and penetration agents, such as PCA, glycerol, propylene glycol monoethyl ether, carbonates, hydrogen carbonates, guanidine, urea and primary, secondary and tertiary phosphates.
Turbidity agents, such as latex or styrene/acrylamide copolymers.
Pearlescent agents, such as ethylene glycol mono and distearate or PEG-3 distearate.
Substantive dyes, and
Propellants, such as propane-butane mixtures, N₂O, dimethyl ether, CO2, and air.

Furthermore, the agents (a), (b) and/or (c) according to the invention can contain further active substances, auxiliaries and additives, such as non-ionic polymers, for example vinyl pyrrolidone/vinylacrylate copolymers, polyvinyl pyrrolidone, vinylpyrrolidone/vinylacetate copolymers, polyethylene glycols and polysiloxanes; additional silicones such as volatile or non-volatile, straight-chain, branched or cyclic, cross-linked or un-cross-linked poly alkyl siloxanes (such as dimethicones or cyclomethicones), poly aryl siloxanes and/or polyalkyl aryl siloxanes, in particular polysiloxanes having organofunctional groups, such as substituted or unsubstituted amines (amodimethicones), carboxyl, alkoxy and/or hydroxyl groups (dimethicone copolyols), linear polysiloxane (A)-polyoxyalkylene (B) block copolymers, grafted silicone polymers; cationic polymers such as quaternized cellulose ethers, polysiloxanes with quaternary groups, dimethyl diallyl ammonium chloride polymers, acrylamide-dimethyl diallyl ammonium chloride copolymers, dimethylaminoethyl methacrylate-vinyl pyrrolidone copolymers quaternized with diethyl sulphate, vinyl pyrrolidone-imidazolinium-methochloride copolymers, and quaternized polyvinyl alcohol; zwitterionic and amphoteric polymers; anionic polymers such as polyacrylic acids or cross-linked polyacrylic acids; structuring substances such as glucose, maleic acid and lactic acid, hair-conditioning compounds such as phospholipids, for example lecithin and cephalin, dimethyl isosorbide and cyclodextrins; fiber structure-improving active substances, in particular mono, di, and oligosaccharides, for example glucose, galactose, fructose, fruit sugars and lactose; dyes for coloring the agent; anti-dandruff active substances such as piroctone olamine, zinc omadine and climbazole; amino acids and oligopeptides; animal-based and/or plant-based protein hydrolysates and protein hydrolysates in the form of their fatty acid condensation products or optionally anionically or cationically modified derivatives; vegetable oils; light stabilizers and UV blockers; active substances such as panthenol, pantothenic acid, pantolactone. allantoin, pyrrolidone carboxylic acids and salts thereof, and bisabolol; polyphenols, in particular hydroxycinnamic acids, 6,7-dihydroxycoumarin, hydroxybenzoic acids, catechins, tannins, leukoanthocyanidins, anthocyanidins, flavanones, flavones and flavonols; ceramides or pseudoceramides; vitamins, pro-vitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; turbidity agents such as latex, styrene/PVP and styrene/acrylamide copolymers; pearlescent agents such as ethylene glycol mono- and distearate and PEG-3 distearate; pigments as well as propellants such as propane-butane mixtures, N²O, dimethyl ether, CO₂ and air.

A person skilled in the art will select these further substances in accordance with the desired properties of the agents. With regard to further optional components and the used quantities of these components, reference is made expressly to the relevant handbooks known to a person skilled in the art. The additional active substances and auxiliaries are used in the agents according to the invention preferably in quantities of, in each case, from 0.0001 to 25% by weight, in particular from 0.0005 to 15% by weight, based on the total weight of the application mixture.

Decolorization of Dyed Keratin Fibers

The multi-component packaging unit according to the invention is a system comprising the agents (a) and (b) (and optionally additionally (c)), which is used for the decolorization of previously dyed keratinic fibers, in particular human hair. The dyed keratinic fibers are usually fibers that have been previously dyed using conventional oxidation dyes and/or substantive dyes known to a person skilled in the art.

The decolorizing agents are suitable for removing colors produced on the keratin fibers using oxidation dyes on the basis of developer and coupler components. If the following compounds were used as developers, the colors produced as a result can be well removed effectively and practically without subsequent darkening using the decolorizing agent: p-phenylenediamine, p-toluenediamine N,N-bis-(β-hydroxyethyl)-p-phenylenediamine, 4-N,N-bis-(β-hydroxyethyl)-amino-2-methylaniline, 2-(β-hydroxyethyl)-p-phenylenediamine, 2-(α,β-dihydroxyethyl)-p-phenylenediamine, 2-hydroxymethyl-p-phenylenediamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-diamino-1-(β-hydroxyethyl)-pyrazole.

If the following compounds were used as couplers, the colors produced as a result can likewise be well removed with very good decolorizing result: m-phenylenediamine derivatives, naphthols, resorcinol and resorcinol derivatives, pyrazolones and m-aminophenol derivatives The following are particularly suitable as coupler substances 1-naphthol, 1,5-, 2,7- and 1,7-dihydroxynaphthaline, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2′,4′-diaminophenoxy) propane, 2-chloro-resorcinol, 4-chloro-resorcinol, 2-chloro-6-methyl-3-aminophenol, 2-amino-3-hydroxypyridine, 2-methylresorcinol, 5-methylresorcinol and 2-methyl-4-chloro-5-aminophenol and 2,6-dihydroxy-3,4-dimethylpyridine.

The substrate to be decolorized can also have been colored using substantive dyes. Substantive dyes include, in particular, nitrophenylenediamine, nitroaminophenols, azo dyes, anthraquinones or indophenols. By way of example, keratin fibers that have been colored by the following dyes known under the following international names or trade names can be decolorized using the multi-component packaging unit (kit-of-parts) according to the invention: HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange ₃, Acid Orange 7, HC Red 1, HC Red ₃, HC Red 10, HC Red 11, HC Red 13, Acid Red 33, Acid Red 52, HC Red BN, Pigment Red 57:1, HC Blue 2, HC Blue 12, Disperse Blue ₃, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamin-2′-carboxylic acid, 6-nitro-1,2,3,4-tetrahydroquinoxaline, 2-hydroxy-1,4-naphthoquinone, picraminic acid and salts thereof, 2-amino-6-chloro-4-nitrophenol, 4-ethylamino-3-nitrobenzoic acid and 2-chloro-6-ethylamino-1-hydroxy-4-nitrobenzene.

Furthermore, the substrates to be decolorized can also be dyed using natural dyes occurring in nature, as are contained for example in henna red, henna neutral, henna black, chamomile blossom, sandalwood, black tea, cascara bark, sage, logwood, madder root, catechu, sedr, and alkanet root.

The decolorizing agents according to the invention are intended for the removal of these dyed colors and therefore themselves preferably do not contain any dyes, i.e. no oxidation dye precursors of the developer type or of the coupler type, and also no substantive dyes.

In a further preferred embodiment a multi-component packaging unit (kit-of-parts) according to the invention is therefore characterized in that
the total quantity of all dyes and oxidation dye precursors contained in the agent (a) lies at a value of at most 0.2% by weight, preferably of at most 0.1% by weight, more preferably of at most 0.05% by weight, and particularly preferably of at most 0.01% by weight—in relation to the total weight of the agent (a),
the total quantity of all dyes and oxidation dye precursors contained in the agent (b) lies at a value of at most 0.2% by weight, preferably of at most 0.1% by weight, more preferably of at most 0.05% by weight, and particularly preferably of at most 0.01% by weight—in relation to the total weight of the agent (b),
the total quantity of all dyes and oxidation dye precursors contained in the agent (c) lies at a value of at most 0.2% by weight, preferably of at most 0.1% by weight, more preferably of at most 0.05% by weight, and particularly preferably of at most 0.01% by weight—in relation to the total weight of the agent (c).

All specified quantities relate here to the total quantity of all dyes contained in the particular agent from the group of substantive dyes, oxidation dye precursors, and natural dyes, and this is set in relation to the total weight of the particular agent.

Mixing Ratio of Agents (a) and (b)

As already described previously, the ready-to-use decolorizing agent is produced by mixing the agents (a) and (b). The agents (a) and (b) can be mixed here primarily in different mixing ratios, for example (a)/(b) of from 1:100 to 100:1.

In order to ensure comfortable mixing, it may be advantageous to use the two agents (a) and (b) in approximately equal quantities. In particular if acids in concentrated form are used in the agent (b), it may also be advantageous to use the agent (a) in a surplus. If, however, the agent (a) is formulated so as to be anhydrous, it may also be advantageous on the other hand to use the agent (b) in a surplus.

The agent (a) contains the reducing agent(s) (a1) preferably in a total amount of from 25.0 to 100% by weight, preferably from 60.0 to 100% by weight, more preferably from 70.0 to 100% by weight, more preferably from 80.0-100% by weight, and particularly preferably from 90.0 to 100% by weight—in relation to the total weight of the agent (a). The reducing agent(s) is/are therefore present in the agent (a) in a relatively concentrated manner.

The use of a surplus of agent (b) is desirable especially when the reducing agents are also used in the agent (a) in these concentration ranges.

In a further preferred embodiment a multi-component packaging unit according to the invention is therefore characterized in that the quantities of the agent (a) in container (A) and of the agent (b) in container (B) are selected such that, when producing the application mixture—i.e. when mixing the agents (a) and (b)—the mixing ratio (a)/(b) lies at a value of from 1:99 to 30:70, preferably 1:99 to 20:80, more preferably from 1:99 to 15:85, and very particularly preferably from 1:99 to 10:90.

At a ratio (a)/(b) of 10:90, 10 parts by weight of the agent (a) are mixed with 90 parts by weight of the agent (b).

In a further particularly preferred embodiment a multi-component packaging unit according to the invention is therefore characterized in that the quantities of the agent (a) in container (A) and of the agent (b) in container (B) are selected such that, when producing the application mixture—i.e. when mixing the agents (a) and (b)—the mixing ratio (a)/(b) lies at a value of from 1:99 to 30:70, preferably 1:99 to 20:80, more preferably from 1:99 to 15:85, and very particularly preferably from 1:99 to 10:90.

In order to produce the mixture, the agent (a) can be transferred completely from container (A) into container (B)—which already contains the agent (b). In this case the size of the container (B) is selected such that the container (B) can receive the total quantity of the agents (a) and (b) and also allows the two agents (a) and (b) to be mixed, for example by shaking or stirring.

Similarly, the mixture can also be produced by completely transferring the agent (b) from container (B) into container (A)—which already contains the agent (a). In this case the size of the container (A) should be selected such that the container (A) can receive the total quantity of the agents (a) and (b) and also allows the two agents (a) and (b) to be mixed, for example by shaking or stirring.

A further possibility for producing the application mixture is the complete transfer of both agents (a) and (b) from the containers (A) and (B) into a third container, which then allows both agents to be mixed—for example by shaking or stirring.

Method

The previously described multi-component packaging units (kits-of-parts) according to the invention can be used in methods for reductive decolorization.

A second subject of the present invention is therefore a method for the reductive decolorization of dyed keratinic fibers, comprising the following steps in the specified order
(I) producing a ready-to-use decolorizing agent by mixing an agent (a) with an agent (b), wherein
   the agent (a) is an agent as defined in the description of the first subject of the invention,
   the agent (b) is an agent as defined in the description of the first subject of the invention,
(II) applying the ready-to-use decolorizing agent to dyed keratinic fibers,
(III) leaving the decolorizing agent to act for a period of time lasting from 5 to 60 minutes, preferably from 10 to 55 minutes, more preferably from 20 to 50 minutes, and particularly preferably from 30 to 45 minutes,
(IV) rinsing off the decolorizing agent from the keratinic fibers.

Steps (I), (II), (III) and (IV) of the method constitute the process of decolorizing the keratin fibers and are consequently carried out in direct chronological sequence successively.

A very particularly preferred embodiment according to the invention is characterized in that the agents (a) and (b) in step (I) are mixed in a proportion of (a)/(b) of from 1:99 to 30:70, preferably 1:99 to 20:80, more preferably from 1:99 to 15:85, and very particularly preferably from 1:99 to 10:90.

The method according to the invention is particularly effective in the case of keratin fibers which have been dyed using certain oxidation dye precursors.

Good results have been attained particularly when the decolorization method is applied to keratin fibers that have been dyed using one or more oxidation dye precursors from the group composed of p-phenylenediamine, p-toluenediamine, N,N-bis-(β-hydroxyethyl)-p-phenylenediamine, 2-(β-hydroxyethyl)-p-phenylenediamine and/or 2-(methoxymethyl)-p-phenylenediamine.

A preferred method according to the invention is therefore also characterized in that the ready-to-use decolorizing agent is applied to keratinic fibers that have been dyed using at least one oxidation dye precursor from the group composed of p-phenylenediamine, p-toluenediamine, N,N-bis-(β-hydroxyethyl)-p-phenylenediamine, 2-(β-hydroxyethyl)-p-phenylenediamine and/or 2-(methoxymethyl)-p-phenylenediamine.

That said in relation to the multi-component packaging unit applies mutatis mutandis with regard to further preferred embodiments of the method according to the invention.

### EXAMPLES

1.1. Coloring

The following formulations were produced (all values in % by weight):

Coloring Cream (F1)

| | % by |
|---|---|
| Raw material | weight |
| Cetearyl alcohol | 8.5 |
| C12-C18 fatty alcohols | 3.0 |
| Ceteareth-20 | 0.5 |
| Ceteareth-12 | 0.5 |
| Plantacare 1200 UP (Laurylglucoside, 50-53% aqueous solution) | 2.0 |
| Sodium laureth-6 carboxylate (21% aqueous solution) | 10.0 |
| Sodiummyreth sulphate (68-73% aqueous solution) | 2.8 |
| Sodium acrylate, trimethylammoniopropylacrylamide chloride | 3.8 |
| copolymer | |
| (19-21% aqueous solution) | |
| Potassium hydroxide | 0.83 |
| p-toluenediamine, sulphate | 2.25 |
| m-aminophenol | 0.075 |
| 2-amino-3-hydroxypyridine | 0.12 |
| Resorcinol | 0.62 |
| 4-chlororesorcinol | 0.26 |
| 3-amino-2-methylamino-6-methoxypyridine | 0.04 |
| 1,3-bis(2,4-diaminophenoxy)propane, tetrahydrochloride | 0.05 |
| Ammonium sulphate | 0.1 |
| Sodium sulphite | 0.4 |
| Ascorbic acid | 0.1 |
| 1-hydroxyethan-1,1-diphosphonic acid (60% aqueous solution) | 0.2 |
| Ammonia (25% aqueous solution) | 7.2 |
| Water | to 100 |

Oxidizing Agent (Ox)

| Raw material | % by weight |
|---|---|
| Sodium benzoate | 0.04 |
| Dipicolinic acid | 0.1 |
| Disodiumpyrophosphate | 0.1 |
| Potassium hydroxide | 0.09 |
| 1,2-propylene glycol | 1.0 |
| 1-hydroxyethan-1,1-diphosphonic acid | 0.25 |
| (60% aqueous solution) | |
| Paraffinum liquidum | 0.30 |
| Steartrimonium chloride | 0.39 |
| Cetearyl alcohol | 3.4 |
| Ceteareth-20 | 1.0 |
| Hydrogen peroxide (50% aqueous solution) | 12.0 |

The coloring cream (F1) and the oxidizing agent (Ox) were mixed in a proportion of 1:1 and applied to strands of hair (Kerling natural European hair, white). The ratio by weight of application mixture:hair was 4:1, and the reaction time was 30 minutes at a temperature of 32 degrees Celsius. The strands were then rinsed with water, dried, and left to rest for at least 24 hours at room temperature. The strands were dyed to a dark brown shade.

1.2. Decolorization

The following decolorizing agents were produced:

Agent (a)

| | | (a) E |
|---|---|---|
| | (a) V | according to the |
| | comparison | invention |
| Sodium dithionite | 5.0 g | — |
| Formamidine sulfinic acid | — | 5.0 g |

Agent (b)

| | (b1) | (b2) |
|---|---|---|
| Cetearyl alcohol | 2.9 g | 2.9 g |
| PEG-40 castor oil | 0.55 g | 0.55 g |
| Sodium cetearyl sulphate | 0.28 g | 0.28 g |
| Hydroxyethan-1,1-disposphonic acid | 0.24 g | — |
| (1-etidronic acid) | | |
| Monoethanolamine | — | 0.95 |
| Water (dist.) | to 95 g | to 95 g |

Agent (b)

| | (b3) | (b4) |
|---|---|---|
| Natrosol 250 (hydroxyethylcellulose) | 1.9 | 1.9 |
| Hydroxyethan-1,1-disposphonic acid | 0.24 g | — |
| (1-etidronic acid) | | |
| Monoethanolamine | — | 0.95 |
| Water (dist.) | to 95 g | to 95 g |

The agent (a)V was mixed with each of the agents (b1) and (b3) (5 g agent(a) and 95 g agent(b))
(a)V+(b1): comparison, decolorizing agent with sodium dithionite, emulsion
(a)V+(b3): comparison, decolorizing agent with sodium dithionite, gel

The agent (a)E was mixed with each of the agents (b2) and (b4) (5 g agent (a) and 95 g agent (b))
(a)E+(b2): according to the invention, decolorizing agent with formamidine sulfinic acid, emulsion
(a)E+(b4): according to the invention, decolorizing agent with formamidine sulfinic acid, gel

These ready-to-use decolorizing agents were each applied to the hair colored under point 1.1 and left to take effect for 30 minutes at a temperature of 30° C.

During the application, the development of any odor was assessed for each decolorizing agent by 10 trained individuals on the basis of a scale from 1 to 4. The average value of the 10 individual values was calculated in each case.

1=very weak odor 4=very strong odor

| | odor |
|---|---|
| (a)V + (b1): comparison, decolorizing agent | 3 |
| with sodium dithionite, emulsion | |
| (a)V + (b3): comparison, decolorizing agent | 4 |
| with sodium dithionite, gel | |
| (a)E + (b2): according to the invention, | 1 |
| decolorizing agent with formamidine | |
| sulfinic acid, emulsion | |
| (a)E + (b4): according to the invention, | 2 |
| decolorizing agent with formamidine | |
| sulfinic acid, gel | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A multi-component packaging unit (kit-of-parts) for the reductive decolorization of dyed keratinic fibers, which unit comprises, packaged separately from one another
a container (A) containing a solid or powdered cosmetic agent (a) and
a container (B) containing an aqueous cosmetic agent (b),
wherein
the agent (a) in container (A)
(a1) includes one or more sulfinic acid derivatives selected from the group consisting of
(H₂N)(NH)C(SO₂H) formamidine sulfinic acid,
HN(CH₂SO₂Na)₂, disodium [(sulfinatomethyl)amino]methane sulfinate
HN(CH₂SO₂K)₂, dipotassium [(sulfinatomethyl)amino]methane sulfinate
HN(CH₂SO₂H)₂, [(sulfinomethyl)amino]methanesulfinic acid
N(CH₂SO₂Na)₃, trisodium [bis(sulfinatomethyl)amino]methane sulfinate
N(CH₂SO₂K)₃, tripotassium [bis(sulfinatomethyl)amino]methane sulfinate
N(CH₂SO₂H)₃, [bis(sulfinomethyl)amino]methanesulfinic acid
H₂NCH(CH₃)SO₂Na, sodium 1-aminoethan-1-sulfinate
H₂NCH(CH₃)SO₂K, potassium 1-aminoethan 1-sulfinate
H₂NCH(CH₃)SO₂H, 1-aminoethan-1-sulfinic acid,
HN(CH(CH₃)SO₂Na)₂, disodium 1-[(1-sulfinatoethyl)amino]ethan-1-sulfinate
HN(CH(CH₃)SO₂K)₂, dipotassium 1-[(1-sulfinatoethyl)amino]ethan-1-sulfinate
HN(CH(CH₃)SO₂H)₂, 1-[(1-sulfinoethyl)amino]ethan-1-sulfinic acid
N(CH(CH₃)SO₂Na)₃, trisodium 1-[bis(1-sulfinatoethyl)amino]ethan-1-sulfinate
N(CH(CH₃)SO₂K)₃, tripotassium 1-[bis(1-sulfinatoethyl)amino]ethan-1-sulinate and
N(CH(CH₃)SO₂H)₃, 1-[bis(1-sulfinoethyl)amino]ethan-1-sulfinic acid,
the total content of all metal salts included in the agent (a), and selected from the group consisting of magnesium salts, zinc salts, iron salts, copper salts, and calcium salts,—in relation to the total weight of the agent (a)—lies at a value below 0.1% by weight, and
the total content of all metal salts included in the agent (b), and selected from the group of magnesium salts, zinc salts, iron salts, copper salts, and calcium salts,—in relation to the total weight of the agent (b)—lies at a value below 0.1% by weight.

2. The multi-component packaging unit (kit-of-parts) according to claim 1, wherein the agent (a) in container (A)
(a1) includes (H₂N)(NH)C(SO₂H) formamidine sulfinic acid as the sulfinic acid derivative.

3. The multi-component packaging unit (kit-of-parts) according to claim 1, wherein the agent (a) in container (A) includes—in relation to the total weight of the agent (a)—one or more sulfinic acid derivatives selected from the group (a1) in a total quantity of from 60.0 to 100.0% by weight.

4. The multi-component packaging unit (kit-of-parts) according to claim 1, wherein
the total quantity of all metal salts included in the agent (a) from the group of magnesium salts, zinc salts, iron salts, copper salts, and calcium salts—in relation to the total weight of the agent (a)—lies at a value below 0.01% by weight, and
the total quantity of all metal salts contained in the agent (b) from the group of magnesium salts, zinc salts, iron salts, copper salts, and calcium salts—in relation to the total weight of the agent (a)—lies at a value below 0.01% by weight.

5. The multi-component packaging unit (kit-of-parts) according to claim 1, wherein the agent (a) in container (A) further includes
(a2) one or more dissolution accelerators selected from the group consisting of homopolymers of (meth)acrylic acid, copolymers of (meth)acrylic acid, polyvinylpyrrolidone (PVP), celluloses, cellulose derivatives, starch, starch derivatives, alginates and casein derivatives.

6. The multi-component packaging unit (kit-of-parts) according to claim 1, wherein the agent (a) in container (A) further includes
(a3) particulate silicon dioxide as an anticaking agent.

7. The multi-component packaging unit (kit-of-parts) according to claim 1, further including—in relation to the total weight of the agent (a)—
additional reducing agents included in the agent (a) selected from the group consisting of sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, thioglycolic acid, and all physiologically acceptable salts of these compounds, wherein the additional reducing agents are included at a value below 0.5% by weight.

8. The multi-component packaging unit (kit-of-parts) according to claim 1, wherein the agent (b) in container (B) has—in relation to the total weight of the agent (b)—
(b1) a water content of at least 60.0% by weight.

9. The multi-component packaging unit (kit-of-parts) according to claim 1, wherein the agent (b) in container (B) has a pH value which lies in a range of from 7.5 to 12.0.

10. The multi-component packaging unit (kit-of-parts) according to claim 1, wherein the agent (b) further includes at least one alkalizing agent selected from the group consisting of monoethanolamine, triethanolamine, 2-amino-2-methyl-propanol, arginine, lysine and histidine.

11. The multi-component packaging unit (kit-of-parts) according claim 1, further including—in relation to the total weight of the agent (b)—
additional reducing agents included in the agent (b) and selected from the group consisting of sodium sulphite, sodium hydrogen sulphite, potassium sulphite, potassium hydrogen sulphite, ammonium sulphite, hydroxy methanesulfinic acid, aminomethanesulfinic acid, cysteine, thiolactic acid, thioglycolic acid, and all physiologically acceptable salts of these compounds, the additional reducing agent being included at a value below 0.5% by weight.

12. The multi-component packaging unit (kit-of-parts) according claim 1, wherein the agent (b) in container (B) additionally includes at least one anionic surfactant.

13. The multi-component packaging unit (kit-of-parts) according to any claim 12, wherein—in relation to the total weight of the agent (b)—the at least one anionic surfactant is included in a total quantity of from 0.2 to 8.0% by weight.

14. The multi-component packaging unit (kit-of-parts) according to claim 1, wherein the agent (b) in container (B) further includes at least one C₁₂-C₃₀ fatty alcohol.

15. The multi-component packaging unit (kit-of-parts) according to claim 1, wherein
the agent (b) includes hydrogen peroxide—in relation to the total weight of the agent (b)—at a value below 1.0% by weight.

16. The multi-component packaging unit (kit-of-parts) according to claim 1, wherein the unit further includes, packaged separately from the other containers,
a container (C) containing a cosmetic agent (c) that includes one or more metal salts selected from the group consisting of magnesium salts, zinc salts, iron salts, copper salts, and calcium salts at a concentration—in relation to the total weight of the agent (c)—at a value below 0.1% by weight.

17. A method for the reductive decolorization of dyed keratinic fibers, including the following steps in the specified order
(I) producing a ready-to-use decolorizing agent by mixing an agent (a) with an agent (b), wherein
the agents (a) and (b) are defined in claim 1,
(II) applying the ready-to-use decolorizing agent to dyed keratinic fibers,
(III) leaving the decolorizing agent to act for a period of time lasting from 5 to 60 minutes, and
(IV) rinsing off the decolorizing agent from the keratinic fibers.

18. The method according to claim 17, wherein the agents (a) and (b) in step (I) are mixed in a proportion of (a)/(b) of from 1:99 to 30:70.
